(19) United States
(12) Patent Application Publication
(10) Pub. No.: US 2016/0022223 A1
(43) Pub. Date: Jan. 28, 2016

(54) MULTI-MODAL DEPTH-RESOLVED TISSUE STATUS MONITOR

(71) Applicant: THE REGENTS OF THE UNIVERSITY OF CALIFORNIA, Oakland, CA (US)

(72) Inventors: Warren S. Grundfest, Los Angeles, CA (US); Marko N. Kostic, Portage, MI (US)

(21) Appl. No.: 14/776,152

(22) PCT Filed: Mar. 12, 2014
(86) PCT No.: PCT/US14/24242

§ 371 Date: Sep. 14, 2015

Related U.S. Application Data
(60) Provisional application No. 61/780,201, filed on Mar. 13, 2013.

(51) Int. Cl. A61B 5/00; A61B 5/03; A61B 5/1455; A61B 5/0285; A61B 5/107; A61B 5/145; A61B 5/0205

(52) U.S. Cl. CPC A61B 5/7278; A61B 5/742; A61B 5/6801; A61B 5/7225; A61B 5/6832; A61B 5/14546; A61B 5/03; A61B 5/02055; A61B 5/0285; A61B 5/1079; A61B 5/14552; A61B 2562/164; A61B 2560/0214; A61B 2560/0223

(57) ABSTRACT

The properties inside a human tissue as well as how those properties vary over time can include information of great importance to a healthcare provider. In some cases, the tissue of interest may not be easily accessible, as a tissue that is under a cast or beneath a bandage, or may be beneath a layer of skin that makes it difficult to evaluate the tissue visually or in a non-invasive manner. The systems and methods described herein relate to monitoring tissue at a plurality of depths.

### CROSS-REFERENCE TO RELATED APPLICATIONS

This application claims the benefit of and priority to U.S. Provisional Patent Application Ser. No. 61/780,201, filed 13 Mar. 2013, which is hereby incorporated herein by reference in its entirety.

### BACKGROUND

The properties inside a human tissue as well as how those properties vary over time can include information of great importance to a healthcare provider. For example, the concentration of hemoglobin, oxygenated or nonoxygenated, blood flow velocity, body temperature, and even change in size of the tissue, can all be relevant to a doctor's understanding of how a wound is healing. In some cases, the tissue of interest may not be easily accessible, as a tissue that is under a cast or beneath a bandage, or may be beneath a layer of skin that makes it difficult to evaluate the tissue visually or in a non-invasive manner. Improved systems and methods for evaluating and monitoring tissues are needed.

### SUMMARY

Systems and methods for monitoring a condition of a tissue are disclosed.

Some embodiments can described as follows:

A system for monitoring tissue at a plurality of depths can include a sensor strip, a data acquisition module and analysis software. The sensor strip can have a first side including a first photodetector element and a plurality of light-emitting elements, wherein the plurality of light-emitting elements are disposed in a predetermined configuration relative to the photodetector element. The data acquisition module can be capable of being coupled to the sensor strip, wherein the data acquisition module is configured to control the sensor strip and store signals received from the light-emitting elements. The analysis software can analyze and/or display the received signals. The system can be adapted to be placed on the surface of a patient's skin, e.g., under a cast, splint, or dressing. The sensor strip can be adapted to be placed over an area of a patient's body, e.g., that has suffered trauma.

In some embodiments, such systems can also include an analog-to-digital converter (ADC), wherein the system differentiates signals received from the light-emitting elements by using the ADC in conjunction with a first photodetector element, and activating only a subset (e.g., one) of the plurality of light-emitting elements at any single point in time.

In some embodiments, such systems can include processing circuitry configured to modulate and demodulate light emitted by the plurality of light-emitting elements.

In some embodiments, the data acquisition module can include a sensor strip control unit configured to control the plurality of light-emitting elements and the first photodetector element. The sensor strip control unit can be configured to generate a modulation sequence for each of the plurality of light-emitting elements that can be differentiated from the modulation sequence for each of the other light-emitting elements activated simultaneously with that light-emitting element.

In some embodiments, a first photodetector element can be configured to detect only a specific wavelength that matches a wavelength of one or more of the plurality of light-emitting elements. In some such systems, all of the light-emitting elements emit substantially the same wavelength of light, or emit light across substantially the same range of wavelengths, or across overlapping ranges of wavelengths. In some such systems each of the plurality of light-emitting elements emits a different wavelength of light, or emits different ranges of wavelengths, in some cases, non-overlapping ranges of wavelengths. In some such systems, the light-emitting elements can emit ultraviolet, visible, and/or near-infrared light. Any, some or all of the light emitting elements can be, for example, a light-emitting diode (LED), including a constant current LED.

In some embodiments, such a system can include two or more photodetector elements.

In some embodiments, a wavelength of light emitted by the light-emitting element(s) and detectable by the photodetector(s) can be selected to detect a chromophore of interest to be found in tissue to be monitored. Not all the photodetectors need be capable of detecting light selected to detect the chromophore of interest.

In some embodiments one or more photodetectors can be a photodiode or a phototransistor.

In some embodiments, a sensor strip can include an ultrasound transducer and/or an ultrasound acquisition unit. Such a sensor strip can include a plurality of ultrasound transducers, e.g., wherein each of the plurality of ultrasound transducers emits a different frequency.

In some embodiments, a first side of the sensor strip can include at least one of electrical traces, electrical components, pressure sensors, and stretch sensors. The sensor strip can also or alternatively include an accelerometer, gyroscope, and temperature sensor. The sensor strip can also include one or more of analog signal processing circuitry, signal filtering circuitry, sensor-driving circuitry, analog-to-digital conversion circuitry, power supply circuitry, digital data processing circuitry, and data communication unit. The first side of the sensor strip can include a connector for the data acquisition module.

In some embodiments, the sensor strip can include a flexible substrate, optionally with a biocompatible adhesive. Such films include polyimide films or other similar flexible materials.

In some embodiments, a data acquisition module can include signal-processing circuitry and communication modules. The data acquisition module can be configured by the analysis software. The data acquisition module can include a printed circuit board, battery pack, and/or an enclosure. Such a printed circuit board can include at least one of power supply circuitry, a data communication unit, a wireless module, sensor strip control circuitry, a user interface control unit, and a power on/off control. Such a printed circuit board can include at least one of a data-processing unit, an algorithm for data processing and analysis, embedded control software, and/or a memory unit. Such a printed circuit board can include a connector for the sensor strip allowing the sensor strip to be operably connected to the data acquisition module. Such a printed circuit board can include at least one of a visual status indicator, a visual alarm indicator, and an audio alarm indicator. Such a printed circuit board can include a connector for a battery charger and wired communication.

In some embodiments, analysis software is adapted to: view, download, store, and analyze data from the data acquisition module; or create and upload, into the data acquisition module, a data acquisition configuration file specific to a patient. Such a configuration file can include, for example, a patient number, a length of a recording session, alarm threshold levels, and communication parameters.

In some embodiments, a method of monitoring a patient can include 1) positioning the first side of a sensor strip of a system of any preceding claim adjacent to a tissue of a patient; 2) activating one or more light-emitting elements; 3) detecting light emitted by the activated elements to generate one or more signals representative of a characteristic of the tissue; and 4) processing the signals to determine the characteristic of the tissue. The characteristic of the tissue can include one or more of: oxygenation state, levels of oxygenated and/or deoxygenated hemoglobin, ratio of oxygenated:deoxygenated hemoglobin, total hemoglobin level, carboxyhemoglobin level, tissue saturation, cardiovascular pulse, hypovolemic/hypervolemic states, muscle intracompartmental pressure, temperature, blood flow velocity, and change in size of tissue under observation.

In some embodiments, a calibration pad can be used for calibrating a sensor strip. The sensor strip can have a first side including a photodetector element and a plurality of light-emitting elements. The calibration pad can include a test pattern within the calibration pad or on an exterior surface of the calibration pad, wherein the test pattern can be detected by one or more wavelengths of light. The test pattern can detectable by positioning the sensor strip adjacent to a surface of the calibration pad, activating one or more of the light-emitting elements, detecting light emitted by the activated elements to generate one or more signals representative of a characteristic of the test pattern, and processing the signals to determine the characteristic of the test pattern. Such calibration pads can be used to determine the positions of the light-emitting elements on the sensor strip relative to the photodetector by processing light emitted from the light-emitting elements, the light having interacted with the test pattern before being received by the photodetector element while the sensor strip is in photocommunication with the calibration pad.

In some embodiments, such calibration pads can be part of a kit including the calibration pad with a sensor strip, a data acquisition module and analysis software as described above.

In some embodiments, such a kit can be used for calibration by 1) positioning the first side of the sensor strip adjacent to and in photocommunication with a surface of the calibration pad, 2) activating one or more of the light-emitting elements, 3) detecting, with the first photodetector element, light emitted by the activated one or more light-emitting elements and reflected, refracted, or diffracted by the test pattern, thereby generating one or more signals representative of a characteristic of the test pattern, 4) storing a representation of the signals in the data acquisition module, and 5) by operation of the analysis software, comparing the stored representations to a template, thereby determining one or more response characteristics of the sensor strip. In some such methods, comparing the stored representations to a template can include fitting the stored representations to predetermined signals representative of the test pattern, thereby determining the relative locations of the activated one or more light-emitting elements and the first photodetector.

### BRIEF DESCRIPTION OF THE DRAWINGS

FIG. 1 schematically shows potential paths taken by light propagating through tissue.

FIG. 2 schematically shows the locations of various components on a particular sensor strip having a single photodetector.

FIG. 3 schematically shows the locations of various components on a particular sensor strip having two photodetectors.

FIG. 4 schematically shows the locations of various components on a particular sensor strip having a single photodetector.

FIG. 5 schematically shows various parts that can make up a data acquisition module.

FIG. 6 is a photograph of a particular sensor strip and data acquisition module.

FIG. 7 is a photograph of another particular data acquisition module.

FIG. 8 is a schematic block diagram of a near-infrared spectroscopy (NIRS) system.

### DETAILED DESCRIPTION

Based on the scattering and anisotropy characteristics of tissue, tissue sampling depth is defined by the photon-path-distribution function for photons migrating from a source to a detector on the surface of the skin. Using the assumption that tissue is homogenously scattering medium, the spatial photon distribution function has a banana-like shape. If one considers weak absorption within the tissue, then the banana-like shape of the photon propagation in tissue is approximated by the equation

$$z \approx \sqrt{\{ \frac{1}{8} [ \sqrt{( {( {x}^{3} + {( {r}_{sd} - x )}^{2} )}^{3} + 32 {{x}^{2} ( {r}_{sd} - x )}^{2} )} - {x}^{3} - {( {r}_{sd} - x )}^{2} ] \}} \qquad ( 1 )$$

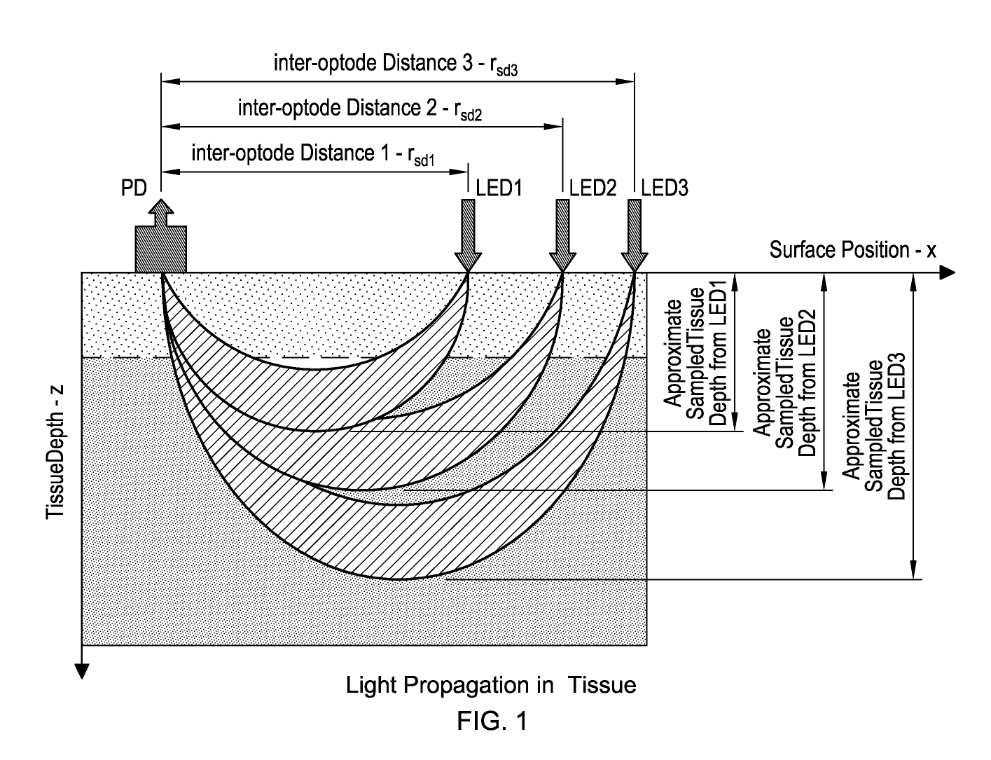

which describes a curve of the most probable direction of photon migration. From FIG. 1 it is evident that the maximum sampled tissue depth, zₘₐₓ, occurs approximately at the mid-point between a light source (e.g., LED1, LED2, LED3) and a light detector (e.g., photodetector PD). Light-emitting diodes LED1, LED2, and LED3 shown in FIG. 1 may or may not be of the same wavelength. Different surface positions of light-emitting elements such as LED1, LED2, and LED3 with respect to a photodetector element affect sampling from different tissue depths.

The distance between a light source and a light detector may be referred to as the inter-optode distance. Therefore, setting the surface position x at the middle of an inter-optode distance, r_{sd}, yields the value of the approximate maximum sampled tissue depth zₘₐₓ, with respect to r_{sd}:

$$at x = \frac{{r}_{sd}}{2} \to {z}_{max} \approx \frac{{r}_{sd}}{2 \sqrt{2}} = 0.354 \cdot {r}_{sd} \qquad ( 2 )$$

where z is tissue depth, r_{sd} is inter-optode distance, and x is surface position.

The present disclosure encompasses a portable, battery-operated, non-invasive, multi-modal, depth-resolved, tissue status monitor. A description of functional testing of an embodiment of such a monitor may be found in the Exemplification section below. Such monitors may include a multi-channel low-power depth-resolved near infrared spectroscopy module, ultrasound module, pressure sensors, temperature sensor, and stretch sensors. These physiological sensors, individually or in various different combinations, are used to obtain depth-resolved information about the tissue health status. Some of the information that may be acquired from the patient to determine tissue health status include, but are not limited to: Oxygenated and deoxygenated hemoglobin concentrations, total hemoglobin, carboxyhemoglobin, tissue saturation, photoplethysmography, onsets of hypo- and hypervolemia states, muscle intracompartmental pressure, body temperature, blood flow velocity, and change in size of tissue under observation.

Some systems and methods of the present disclosure may be used to acquire and analyze signals representative of a physiological quantity, and to inform the clinician about the health status of tissues under observation. In some embodiments, a device is designed for use on the surface of the skin and placed under a cast or splint at the time of surgery to monitor tissue viability. In some embodiments, a patch, such as a lightweight and/or adhesive patch, is placed over an area that has suffered trauma and the patch provides real-time physiologic monitoring data of the affected area and can be used as an acute compartment syndrome detector or tissue flap monitor. Some of the other examples where systems and methods of the present disclosure may be used include, but are not limited to: monitoring of tissue after vascular surgery; monitoring of lower or upper limb tissue viability during prolonged surgeries; or monitoring of skin flaps after mastectomy.

Certain monitors of the present disclosure allow the clinician to obtain depth-resolved information. This is useful, for example, in cases where tissue is very thin or consists of multiple layers. This monitor can be set to allow differentiation of signals from different layers. Technology described herein is also capable of including a variety of other sensor modalities to complement this information.

In some embodiments, a monitor consists of three main components: (1) a sensor strip to be placed on patient skin, the strip containing physiological and other sensors; (2) a data acquisition module, which contains signal processing circuitry as well as storage and communication modules; and (3) analysis software, which can be used to analyze signals collected from the sensor strip, to view and analyze patient data, and to configure the data acquisition module for different recording sessions.

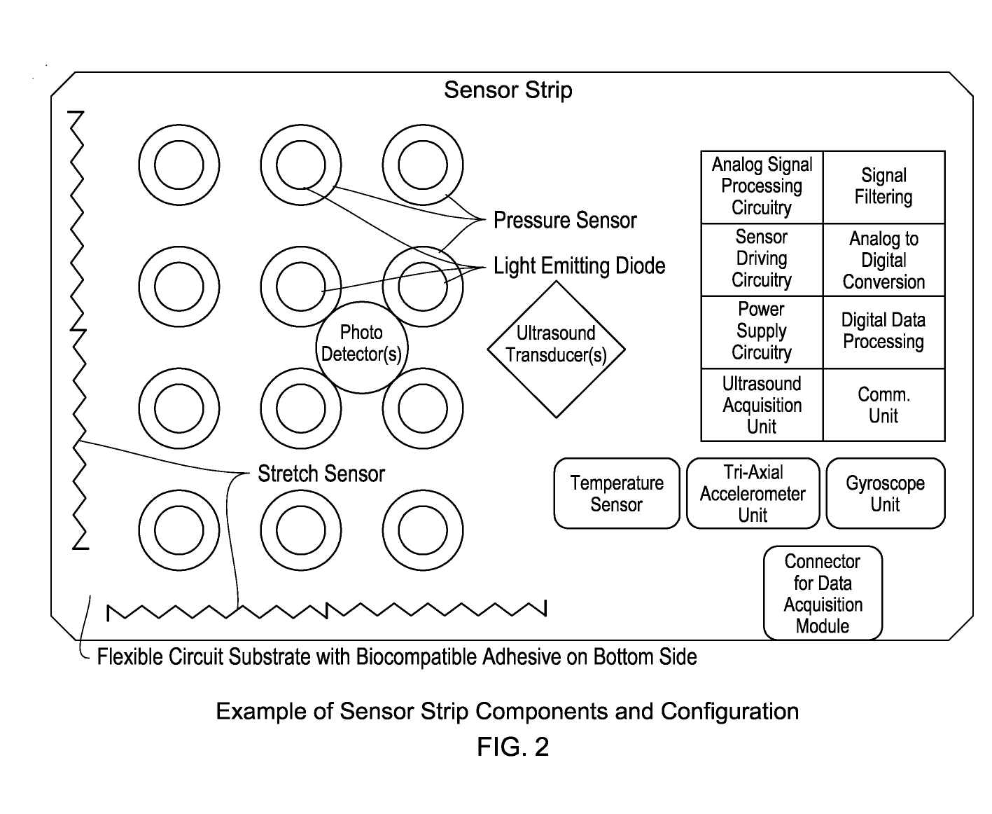

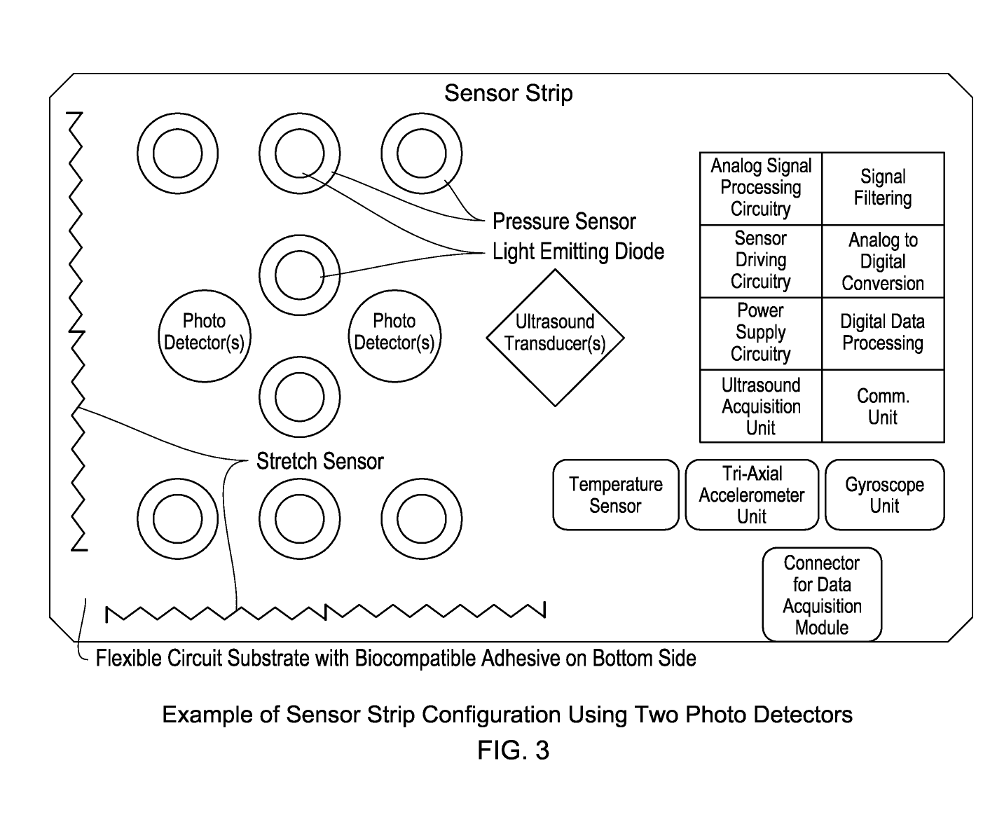

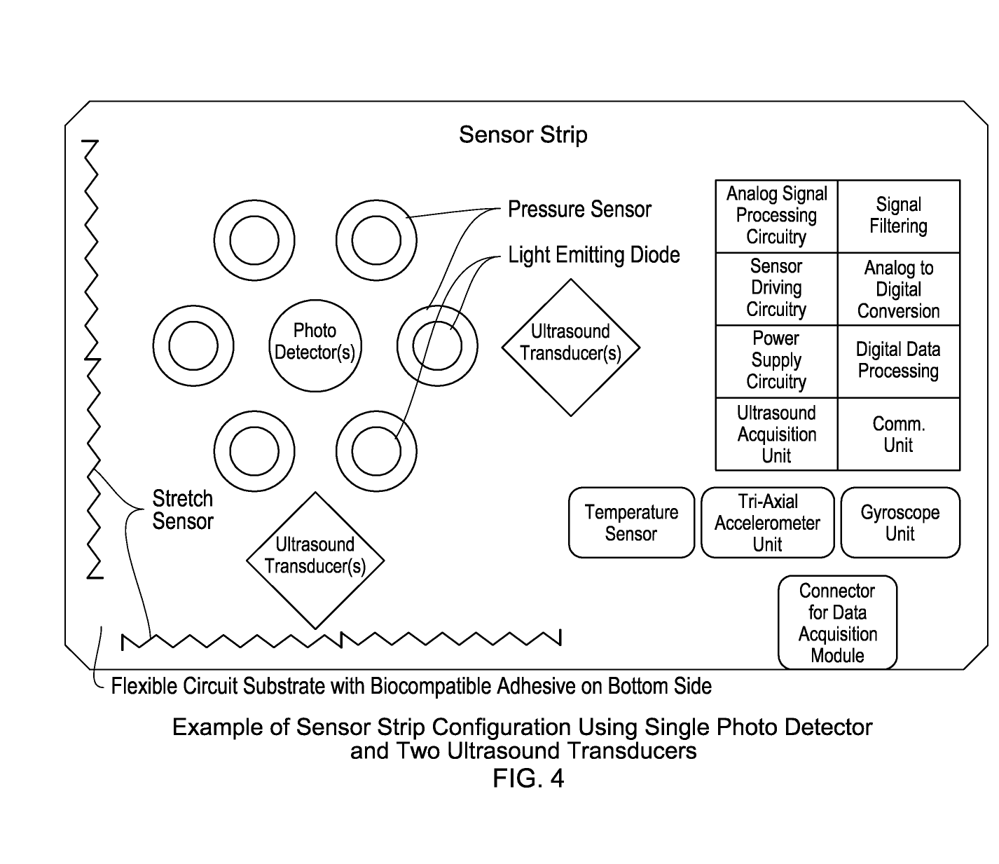

The sensor strip can include a flexible substrate (e.g. polyimide film or similar material) with biocompatible adhesive on bottom side (toward patient skin) and electrical components, sensors, and electrical traces on the opposite side. In some embodiments the sensor strip will contain multiple pressure sensors, light sources (e.g., light-emitting diodes, LEDs), stretch sensors, and one or more photodetectors (e.g., photo diode, photo transistor). FIG. 2 schematically shows a sensor strip with a single photodetector (PD). In various embodiments, single or plural numbers of PDs may be used in different geometric configurations to obtain depth-resolved NIRS information from underlying tissues. Any photodetector capable of detecting the emitted light as it emerges from the tissue can be used. The number of photodetectors and light sources can depend on the clinical application. Examples of different geometric configurations are shown in FIGS. 2-4.

Depth-resolved information may be obtained either using a single photodetector element and multiple light-emitting elements, or with multiple photodetector elements. Embodiments having only a single photodetector typically make use of one or more methods of discriminating between the signals associated with different light-emitting elements. The following are examples of how to effect such discrimination. While some of the following methods apply only to single photodetector embodiments or multiple photodetector embodiments, other methods apply to both.
(1) Only a single light-emitting element is turned ‘ON’ (i.e., emits light) at a single point in time. It may be desirable to convert an analog signal acquired by the photodetector element into a digital signal to facilitate a determination of which light-emitting element corresponds to the acquired signal. Thus, the photodetector element may be used in conjunction with an analog-to-digital converter (ADC). Analog circuitry may be used to process the analog signal acquired by the photodetector element, and the ADC may digitize the analog signal into digital data for further analysis to determine which light-emitting element was ‘ON’ at which time. A sensor strip control unit may be responsible for both emitter and photodetector/ADC control.
(2) Light from the emitters may be modulated and then demodulated by processing circuitry. In this case, each light-emitting element would have its own unique modulation sequence generated by a sensor strip control unit.
(3) Each photodetector element may detect only a specific wavelength that matches a specific emitter wavelength, or a single photodetector element may detect multiple wavelengths and distinguish each source light-emitting element based on the wavelength of the received signal.
(4) Any combination of the above techniques (e.g., turning on a subset of the light-emitting elements, each of the light-emitting elements having a unique modulation sequence relative to the other light-emitting elements activated at the same time; activating subsets of light-emitting elements such that each of the simultaneously-activated light-emitting elements emits a different wavelength; having the some light-emitting elements emit signals of the same wavelength, but using different modulation sequences for different emitters that are operating at the same wavelength; etc.).

Light-emitting elements may be selected based on the clinical application of the monitor. For example, emitters having a particular output (e.g., emitted wavelength), or several emitters collectively having a range of wavelengths, may be selected depending on the specific chromophore of interest that is to be investigated. The selection of light-emitting elements may guide the selection of an appropriate photodetector element or elements. A photodetector element may be selected that best matches the output of the emitters (e.g., a detector that detects a particular wavelength or range of wavelengths), or that best matches only a subset of the emitters. A wide variety of light emitting elements is known in the art, and any appropriate light emitter may be used.

In some embodiments, the sensor strip may include two or more photodetector elements. Multiple emitters and one or more detectors may be used in different configurations depending on the clinical application of the monitor. As explained above, the farther a photodetector is from the light emitting element whose light is being detected, the deeper the maximum tissue depth being probed. By arranging photodetectors and light emitting elements around the sensor, a variety of depths can be probed at a variety of different locations beneath the surface, allowing the user to build three-dimensional information on the nature of the tissue beneath the sensor strip. Many different configurations of light emitters and photodetectors may be useful in different contexts, for example, detectors and emitters could be arranged to probe only a narrow range of depths by over a large area if the tissue to be investigated a relatively shallow, flap-type incision or wound. Or if the tissue is known to include a deep, generally vertical incision or wound, i.e., a cut that is along a plane perpendicular to the exterior surface of the tissue, a sensor strip with emitters and detectors arranged so as to probe a larger variety of depths along a single plane might be preferable.

In some embodiments, the sensor strip may include one or more ultrasound transducers. For certain clinical applications, a single ultrasound transducer may be sufficient. Multiple ultrasound transducers, however, may provide better depth-resolved information compared to a single transducer. For example, each transducer may emit a different frequency in order to preferentially obtain information from different depths of tissue (e.g., higher frequency transducers have shorter penetration depth but better resolution and vice versa). The information from the ultrasound transducer(s) may be used to complement information obtained from light-emitting elements, or may be processed as a stand-alone modality. The ultrasound information is not necessary for operation of the light-emitting elements. The ultrasound transducer module(s) are an optional part of the sensor strip depending on the clinical application of the device.

Additionally, the sensor strip may include a single or plural number of accelerometers, gyroscopes, and temperature sensors, for example as solid state devices such as MEMS. Furthermore, the sensor strip may contain analog signal processing circuitry, signal filtering circuitry, sensor driving circuitry, analog-to-digital conversion circuitry, power supply circuitry, ultrasound acquisition unit, digital data processing circuitry, data communication unit, and connector for being operably connected to a data acquisition module. The sensors and electrical components may be placed in any number of geometric combinations on the sensor strip. Moreover, the information from each sensor may be used individually or in combination with any or all other sensor data to monitor tissue viability, and/or tissue flap status, and detect acute compartment syndrome.

An operable connection between the sensor strip and the data acquisition module can be a wired connection or can be wireless. As with many medical monitors, a wired connection might be convenient where the sensor strip is placed on an in-patient or other person confined to a bed. Wireless connections between the various parts of the system may be preferable where the patient is mobile. However, even for mobile patients, a wired connection may be useful, since the entire system can be designed to be light-weight and easily transportable. Different portions of the system may be designed to be carried on the patient's person. In some embodiments, the sensor strip itself may have a wireless connection to the rest of the system, in which case the patient need only keep the sensor strip. In other embodiments, the sensor strip can be wired to the data acquisition module where signals are stored. Data can then be transferred from the data acquisition module in any number of ways. The data acquisition module can include a wired or wireless connection to a computer on which analysis software can be executed. Or the data acquisition module can store data on a removable memory medium, such as flash memory, which can then be physically removed to a computer that is not otherwise connected to the data acquisition module. Alternatively, the data acquisition module can have a wired or wireless connection directly into a network, such as a LAN, so as to transmit received and stored data in real-time to a computer. In any of the above embodiments, the data can be analyzed and compared to criteria designed to detect one or more pathologies in the patient's tissue. As described in more detail below, the analysis of the data can trigger an alarm if a criterion is met or if a pathology is detected or inferred.

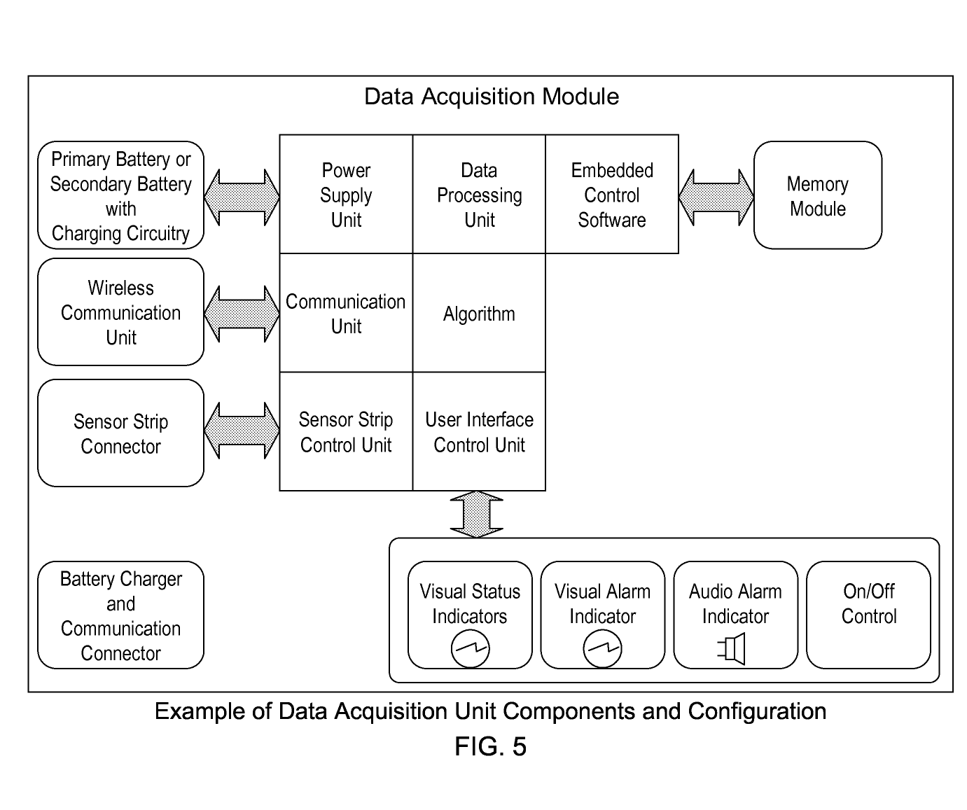

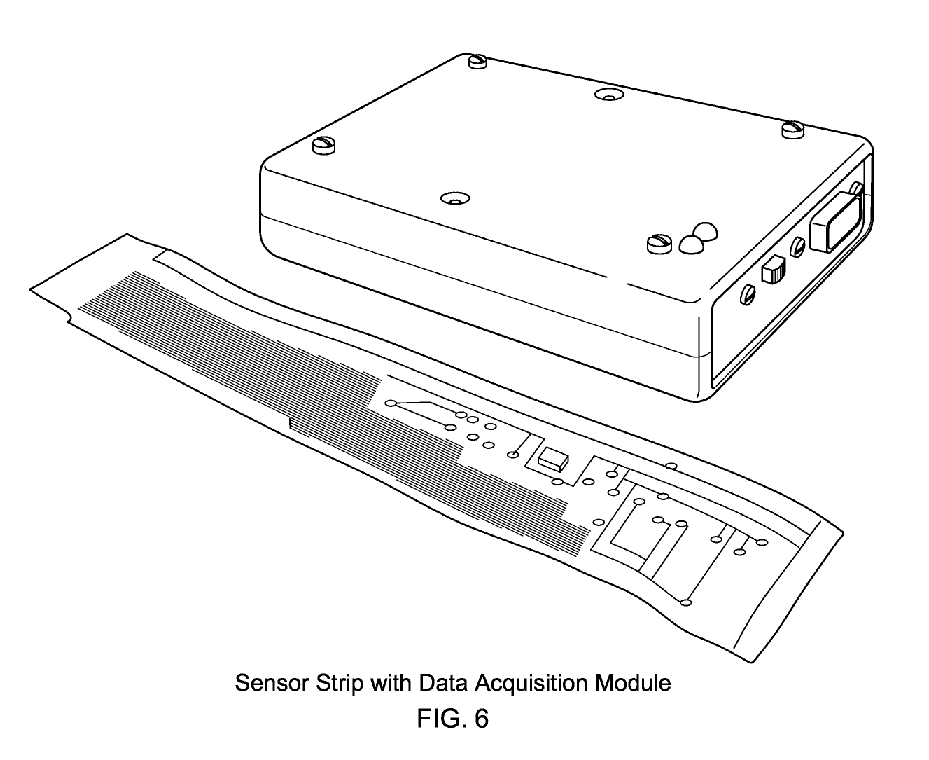

A data acquisition module can include a printed circuit board (flexible or solid), a primary or secondary battery pack, and an enclosure. The printed circuit board can include power supply circuitry (including a battery charger), a data communication unit, a wireless module, sensor strip control circuitry, a user interface control unit, a data processing unit, memory media (e.g., an SD card or other data storage unit, possibly removable), a connector for the sensor strip, a visual status indicator(s), a visual alarm indicator(s), an audio alarm indicator, a power ‘on/off’ control, and/or a connector for battery charger and/or wired communication. Many of the above units, such as the sensor strip control circuitry, the user interface control unit the data processing unit, and the memory media, are capable of storing software. Such stored software can be used, for example, for data processing and/or analysis, or operational control and can include algorithms specific to those or other tasks. FIG. 5 and FIG. 6 show examples of a data acquisition module.

In some embodiments, a personal computer or similar mobile device is provided with analysis software that includes a computer code programmed with a series of instructions that allow a user to view, download, store, and analyze data from the data acquisition module. In addition, software can be used to create and upload one or more data acquisition configuration files specific to each patient into the data acquisition module. The configuration file may contain information such as, but not limited to, patient number, length of the recording session, alarm threshold levels, communication parameters and relevant elements of patient history.

A particular aspect of the present disclosure is the use of a series of emitters and at least one photodetector sensor to obtain depth-resolved information in a substrate, such as living tissue. To ensure stable outputs, the emitters may be constant current LEDs and a detector is chosen to match the outputs of the LEDs. This unique combination of inputs and outputs is combined with geometric placement of the emitters on the sensor strip to achieve differentiation in signals from various tissue layers. We have already validated this in an initial human trial.

Various monitors and systems disclosed herein can be used in at least the following ways:
1. A reusable or single-use sensor strip is attached to the patient skin and a data acquisition module is connected to the strip.
2. A clinician or authorized person powers-up the data acquisition module and loads the appropriate data acquisition configuration file.
3. The data acquisition module initializes and verifies proper state of the sensors embedded in the sensor strip, for example by calibration as explained below.
4. After the successful start-up, the data acquisition module goes into acquisition mode for the duration of session (e.g., according to a predetermined acquisition routine or as determined by the clinician).
5. Data acquired during the session may be stored onto a device-based memory medium for later retrieval and analysis. At the discretion of the clinician, real-time physiological data may be viewed on a designated platform via wireless or wired interface.
6. During data acquisition, the data acquisition module may utilize an embedded processing unit to process the acquired physiological signals and determine if, for example, any of the pre-selected physiological abnormalities or conditions are present in tissues under observation.
   i. If no abnormalities are present, the unit does not alarm.
   ii. If the algorithm determines that there may be an abnormality present, it alarms by either visual, audio, or both means. An optional communication link may be established with a server at a healthcare center that would enable real-time viewing of patient acquired data by trained healthcare providers, or that may send an alarm signal or other appropriate notice to the patient's physician or other healthcare provider.
   iii. For outpatients, if necessary, the monitoring center personnel may contact the patient and instruct them to call their clinician for follow-up or observation, or may contact the patient's physician or other healthcare provider directly.
7. At the end of the data acquisition, data acquisition module finalizes the recorded data file on the local memory medium and then powers-down.
8. The clinician removes the sensor strip from the patient and either discards it (if it is a single-use strip) or disinfects it for the next patient (if a reusable strip).
9. At some point, either before, after or during use on the patient, the sensor strip can be applied to a calibration pad. Data can be recorded, and characteristics of the calibration pad analyzed and compared to a template based on the calibration pad's predetermined characteristics. Differences between the measured and known properties of the calibration pad can then be used to calibrate the data acquired from the patient tissue.

In some embodiments a device or kit includes a sensor strip, data acquisition module and receiver station. The sensor strip can be either reusable or disposable. The device may be used under a cast or dressings to monitor tissue viability. For example, if a patient has a complex lower limb fracture and a clinician is concerned about acute compartment syndrome, the device would be placed over the anterior compartment prior to casting or bandaging. The bandage or cast would be applied as usual and the data acquisition module would be monitored to provide real-time data. Depending on the condition of the patient, monitoring could be in real-time (e.g., continuous) or at various time increments. For inpatients this could be displayed on a monitor. For outpatients who have a cast placed, but are otherwise able to go home, the technology would allow for remote monitoring, for example over the Internet or a telephone line, allowing the clinician to obtain a range of physiologic data remotely. When the cast is removed the device can be recovered.

In some embodiments a calibration pad can be used to verify that the system is working properly before, after and/or interleaved with data collection. A calibration pad can be generally sized and shaped to be complementary to the sensor pad. The calibration pad can include a test pattern in its interior or on its surface. The test pattern can be detectable in one or more wavelengths of light. For example, the calibration pad could have material with a first near infrared chromophore at a first depth and a second, different chromophore at a second different depth. The calibration pad could have a wide variety of materials with different infrared properties throughout its interior and on its surface, e.g., arranged in a two or three dimensional pattern, gradient or other suitable configuration.

The calibration pad can be used by positioning the sensor strip adjacent to the surface of the calibration pad, activating on or more light-emitting elements on the sensor strip, detecting light emitted by the activated light-emitting elements to generate one or more signals representative of the test pattern, and processing the signals to determine a characteristic of the test pattern. The characteristic could be, for example, a particular near infrared spectral response at a first depth within the calibration pad and a second, distinct near infrared spectral response at a second depth, for example on the surface of the calibration pad.

The calibration pad can include a test pattern that is designed to allow for determination of the performance of the sensor pad. The sensor pad can be positioned on the calibration pad with light emitting element(s) and photodetector(s) facing the calibration pad, light emitting elements on the sensor pad activated, emitted light detected by a photodetector or photodetectors on the sensor strip, and the detected light translated in to signals that are transmitted to a data acquisition module or other processor where a representation of the signals is stored. The stored representations can then be compared to a template based on the predetermined properties of the calibration pad, thereby determining one or more response characteristics of the sensor strip, or other component of the above system. Because the test pattern can have a predetermined form, analysis of the signals can be used to determine the location of a photodetector and/or light emitting elements of the sensor pad relative to the test pattern on the calibration pad, and thus to each other. The detected characteristics of the calibration pad can also be used to determine other properties of a photodetector and light emitting elements, such as brightness, sensitivity. A wide variety of characteristics of the system can be characterized and the system calibrated by comparing the known, predetermined properties of the test pattern to how the test pattern is actually detected. Comparing the data collected on the calibration pad to a template of the calibration pad can include, for example, determining how to best fit a predetermined model response function to the data, and inferring from that best fit the properties of the sensor strip and its components and/or other elements of the system.

Once aspects of the sensor strip, such as sensitivity, brightness, and/or relative positions of the various emitters and/or sensors, have been determined in the calibration process, that information can be used by the system to interpret the signals stored by the data acquisition module. As explained above, knowing how far a particular light emitter is from a particular photodetector is important in understanding what depth of tissue is being probed by the detected light. By calibrating the system to a particular sensor strip, the user can allow the software to take into account ordinary variations in the sizes and shapes of sensor strips. Such variations could result from differences within manufacturing tolerances, deformation (e.g., stretching) of the sensor strip over time, or other causes and need not be representative of any sort of defect.

Any of a calibration pad, a sensor strip, a data acquisition module, and relevant software can be combined in a kit. The kit can then be used as explained above to calibrate the response of the sensor strip, data acquisition module and/or software package.

It should be understood that the device of the present disclosure is applicable to all limbs and anywhere where a cast or dressings are placed. This is in addition to other applications mentioned previously (e.g., tissue flaps, vascular surgery, etc.).

The invention now being generally described, it will be more readily understood by reference to the following examples which are included merely for purposes of illustration of certain aspects and embodiments of the present invention, and are not intended to limit the invention.

Exemplification

A light-weight multi-channel multi-wavelength ultra-low power near infrared spectroscopy (NIRS) system was designed and tested. The NIRS system was designed for clinical use to emit low power (maximum 5 mW) red and near-infrared (NIR) light into human tissue and acquire, record, and display reflected light from various tissue depths. As described below, results of initial functional tests of the system are presented. Potential clinical applications of the NIRS system include long-term non-invasive monitoring of functional activity in tissues, oxygen consumption in skeletal muscles, and tissue blood perfusion.

Introduction

Near infrared spectroscopy (NIRS) is a non-invasive, non-ionizing imaging technique that uses light in the 650 nm to 2,500 nm region of the electromagnetic spectrum. In medical applications, optical devices utilize what is known as the biologic window (i.e., “therapeutic window”). This window encompasses the light from 600 nm to approximately 1400 nm. The reason why many medical optical devices exploit light sources within this spectrum is that tissue proteins are relatively transparent at these wavelengths with the exception of certain chromophores such as oxygenated and deoxygenated hemoglobin, melanin, fat, and water. Light is highly scattered by the cells and organelles in tissues, as well as absorbed by certain chromophores. Understanding scattering, absorption, and penetration of light in tissue allows extraction of information from different tissue depths. Modeling tissue scattering and absorption helps analyze light being detected at the surface. Since their introduction, medical NIRS devices have been used in many physiologic monitoring applications, including, pulse oximetry, functional NIR for measuring the neuronal activity in the brain, measurement of oxygen consumption in skeletal muscles, and more recently the measurement of tissue blood perfusion.

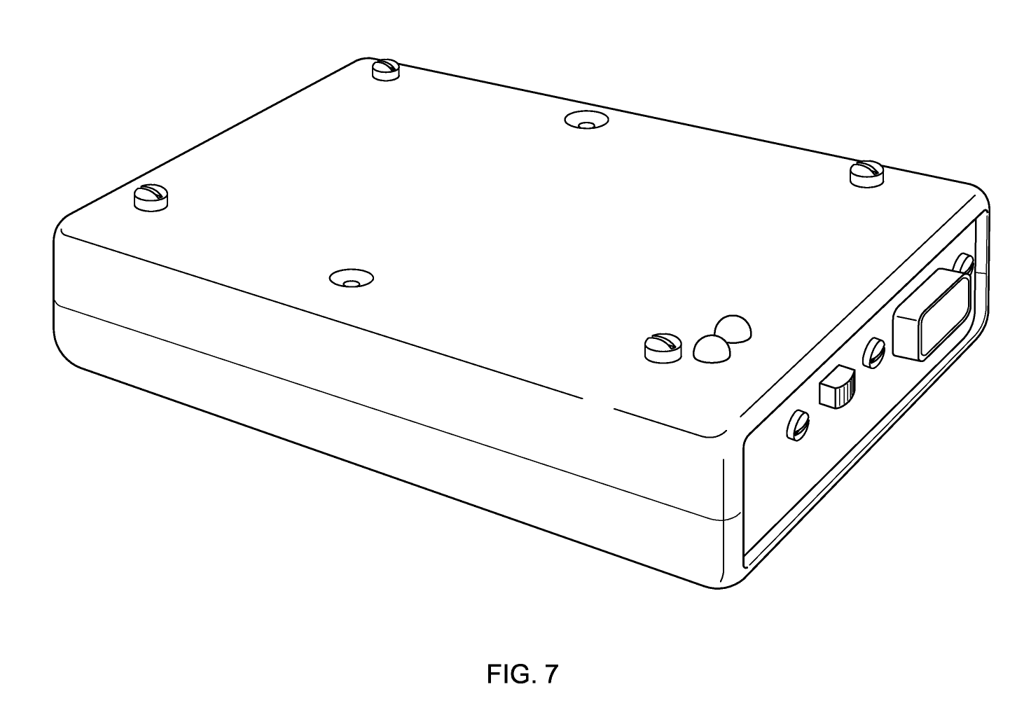

Below, initial functional testing results of a novel multi-channel multi-wavelength ultra-low power portable NIRS system (FIG. 7) are presented. To the best of our knowledge, the capabilities of this device, such as its ability to obtain optical information from multiple depths in tissue from a portable battery powered system for extended periods of time, has not been previously reported. This noninvasive system is designed to emit low-level red and NIR light into human tissue and acquire, record, and display the reflected light from various tissue depths. The level of reflected red and NIR light will vary, primarily, due to absorption by the chromophores of interest and the scattering coefficient of the tissue. The chromophores of interest include HbO₂ and Hb hemoglobin, melanin, fat, water, and lipids.

In preparation for human clinical trials, the objective of this study was to verify several design parameters, including power consumption, sampling rate, total system weight, and real-time multi-channel data display.

1. METHODS & MATERIALS

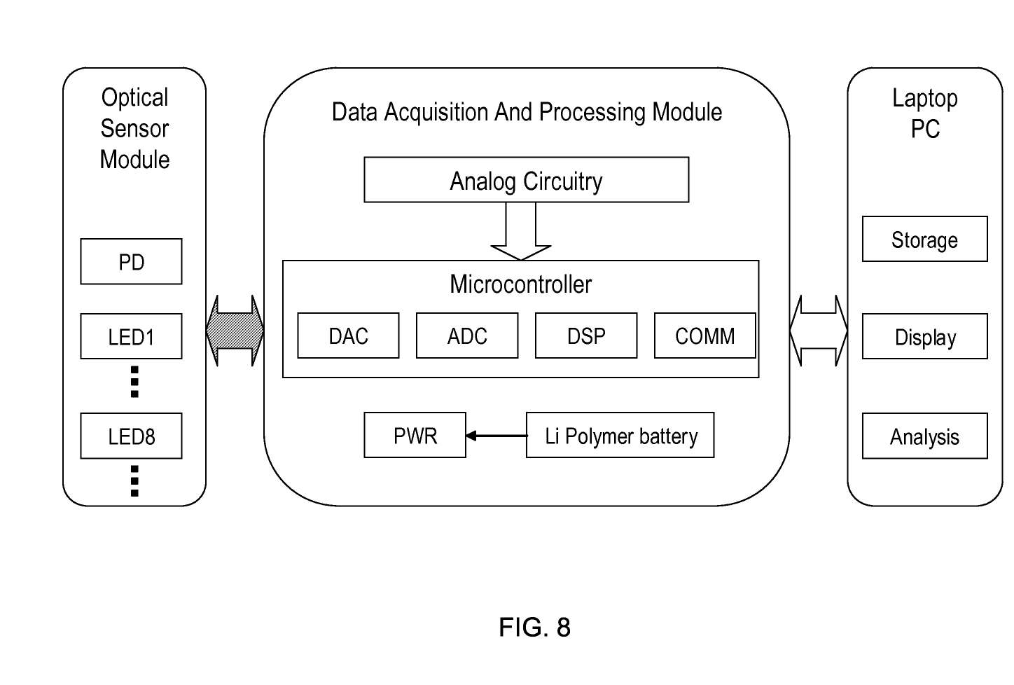

The NIRS system consists of an optical sensor module, data acquisition and processing module, and a PC computer used for real-time data display, analysis, and storage (FIG. 8). These components are described in further detail next.

### 1.1. Hardware

The system consists of a custom-made optical sensor module, data acquisition unit, and a laptop PC. At the heart of the system is an ultra-low power microcontroller, MSP430-family by Texas Instruments. The MSP430 family was selected because of its ultra-low power requirements and processing capabilities.

Based on project requirements and microcontroller capabilities, the MSP430G461x was selected for the initial prototype. This MSP430 device features a 16-bit RISC CPU, a high performance 12 channel 12-bit A/D converter (with 610 μV LSB) and one universal synchronous/asynchronous communication interface (USART). Digitized data is sent to the PC in binary format using the serial communication protocol. Serial communication protocol (i.e., serial port profile, SPP) is one of the most common protocols used for Bluetooth® wireless interface. Finally, the MSP430FG461x series supports a liquid crystal display (LCD) option with its integrated LCD driver.

The system was designed to obtain information about various tissue chromophores at varying tissue depths. This has been achieved by using multiple source-detector distances to collect reflected light. Light obtained from a near source-detector pair samples tissue closer to the surface, while the light obtained from the source-detector pairs several centimeters apart is able to sample deeper sections of tissue. Understanding the results from these optodes requires careful modeling and algorithm development to interpret the data (see below). The optical sensor module contains light sources, LEDs, and a photodetector, PD. The optical signal strength at the detector position on the surface of the skin is expected to be on the order of pico- to micro-watts, which depends on the actual radiant intensity of the source. In our system, we set a goal of generating maximum 5 mW radiant power from LEDs. This value was chosen because it is considered to be a safe optical and thermal level for medical devices. The system utilizes silicone PIN diodes for reflected light detection. The PIN diodes have wide bandwidth, low capacitance, and low bias voltage. Their optical sensitivity is approximately two orders of magnitude smaller than avalanche photo diodes (APDs). Preliminary tests, however, have shown that these detectors have sufficient sensitivity for our applications.

The initial system requirements were based on a need for a fully portable (i.e. light weight), compact multi-channel system capable of 36 hours standby time, 12 hours of continuous NIRS data acquisition at 20 samples per second using 700 mAh rechargeable lithium-polymer battery. The sampling rate was based on the work by Saager, who found that 20 Hz offers more than sufficient sampling rate for characterizing hemodynamic fluctuations, which mostly occur in single- to sub-Hz range. Based on these requirements, the current consumption in the ready (i.e., standby) mode would need to be 19 mA and 58 mA in the active mode. In addition, the system would need to display multi-channel real-time acquired data and save it to the PC hard drive for offline analysis.

1.2. Software

The initial version of the PC software for NIRS data acquisition, display, and storage utilizes custom-designed application developed with Microsoft® DirectX® technology. The application is capable of displaying up to 64 channels of data with various user-configurable parameters such as display scale, signal grouping, and displayed data color. Presently, the acquired data is saved to a local hard drive for off-line analysis. Initial signal processing algorithms have been developed and will be optimized pending the results of our clinical trials.

### 2. RESULTS

Four bench-top tests were conducted to evaluate initial performance of the NIRS system. First, the system current was measured using the ampere meter in the Agilent E3631A triple power supply. The voltage was set to 7.6V DC, and the current was measured in “ready” mode and then in “active” mode. In ready mode, system is set to acquire data with the sensor strip disabled. In active mode, the system is acquiring and sending NTRS data to PC for display and storage. The design goal for the ready mode current was set to 19 mA and was measured to be 16.5 mA, which is approximately 15 percent improvement over the design goal. Active mode current goal was set to 58 mA but was measured to be 60.3 mA. Second, in order to be able to monitor certain physiologic parameters, the system needed to be able to sample acquired optical signals at 20 samples per second (sps). We used Agilent 33120A arbitrary function generator, Agilent DSO1024A oscilloscope, and PC application to test the accuracy of our analog-to-digital conversion, as well as to verify our maximum data sampling rate. The current version of the system is able to acquire NIRS data at a rate of 50 samples per second. Third, total system weight was measured to be 95 grams, which is five grams below design goal. Finally, the last major design goal was achieved by successfully displaying 64 channels of data in real-time. The summary of initial NIRS prototype test results is shown in Table 1. The system succeeded in accomplishing four of the five main goals for this stage of system development. The one parameter that requires further optimization is the active mode current consumption, which exceeded our goal by four percent. The 12 hour continuous active mode operation of the NIRS system will be achieved by making improvements to the embedded control software.

TABLE 1
Design Success Metrics for the NIRS System

| | | Testing | Goal |
|---|---|---|---|
| Parameter | Design Goal | Result | Achieved? |
| Current Consumption (mA) | | | |
| Ready Mode | 19 | 16.5 | Yes |
| Active Mode | 58 | 60.3 | No |
| Sampling Rate per Channel (sps) | 20 | 50 | Yes |
| Total Weight (including battery) [g] | 100 | 95 | Yes |
| Real-time multi-channel data | 64 | 64 | Yes |
| display | | | |

### 3. CONCLUSIONS AND DISCUSSION

The details above describe initial design and functional testing results of a novel multichannel multi-wavelength ultra-low power portable NIRS system. The NIRS technology works by quantifying light absorption by chromophores of interest and the scattering coefficients of the tissue. The clinical applications of this lightweight, multi-channel NIRS system includes long-term non-invasive monitoring of functional activity in tissues, oxygen consumption in skeletal muscles, and tissue blood perfusion.

### INCORPORATION BY REFERENCE

All publications and patents mentioned herein are hereby incorporated by reference in their entirety as if each individual publication or patent was specifically and individually indicated to be incorporated by reference. In case of conflict, the present application, including any definitions herein, will control.

### EQUIVALENTS

Those skilled in the art will recognize, or be able to ascertain using no more than routine experimentation, numerous equivalents to the devices, systems and methods described herein. Such equivalents are considered to be within the scope of this invention and are covered by the following claims. Those skilled in the art will also recognize that all combinations of the various embodiments described herein are within the scope of the invention.

1. A system for monitoring tissue at a plurality of depths, the system comprising:
a sensor strip, the sensor strip having a first side comprising a first photodetector element and a plurality of light-emitting elements, wherein the plurality of light-emitting elements are disposed in a predetermined configuration relative to the photodetector element;
a data acquisition module capable of being coupled to the sensor strip, wherein the data acquisition module is configured to control the sensor strip and store signals received from the light-emitting elements; and
analysis software for analyzing and displaying the received signals.

2. The system of claim 1, wherein the sensor strip is adapted to be placed on the surface of a patient's skin.

3. The system of claim 1, wherein the sensor strip is adapted to be placed over an area of a patient's body.

4. The system of claim 1, further comprising an analog-to-digital converter (ADC), wherein the system differentiates signals received from the light-emitting elements by using the ADC in conjunction with the first photodetector element, and activating only a subset of the plurality of light-emitting elements at any single point in time.

5. The system of claim 1, further comprising processing circuitry configured to modulate and demodulate light emitted by the plurality of light-emitting elements.

6-7. (canceled)

8. The system of claim 1, wherein the first photodetector element is configured to detect only a specific wavelength that matches a wavelength of one of the plurality of light-emitting elements.

9. (canceled)

10. The system of claim 1, wherein each of the plurality of light-emitting elements emits a different wavelength of light.

11. The system of claim 1, wherein the plurality of light-emitting elements emit one or more wavelengths in a spectrum of light including the near-infrared spectrum, the visible spectrum, and the ultraviolet spectrum.

12-14. (canceled)

15. The system of claim 1, wherein one or more wavelengths of the plurality of light-emitting elements are selected based on a chromophore of interest in the tissue.

16. The system of claim 1, wherein the first side of the sensor strip further comprises a second photodetector element, and wherein at least one of the first and second photodetector elements detects one or more wavelengths of light emitted by the plurality of light-emitting elements.

17-22. (canceled)

23. The system of claim 1, wherein a second side of the sensor strip comprises a flexible substrate.

24. The system of claim 1, wherein a second side of the sensor strip comprises a biocompatible adhesive.

25-27. (canceled)

28. The system of claim 1, wherein the data acquisition module comprises a printed circuit board, battery pack, and an enclosure.

29-37. (canceled)

38. A method of monitoring a patient comprising:
1) positioning the first side of a sensor strip of a system of claim 1 adjacent to a tissue of a patient;
2) activating one or more light-emitting elements;
3) detecting light emitted by the activated elements,
4) generating one or more signals representative of a characteristic of the tissue; and
5) processing the signals to determine the characteristic of the tissue.

39. The method of claim 38, wherein the characteristic of the tissue comprises one or more of: oxygenation state, level of oxygenated hemoglobin, level of deoxygenated hemoglobin, ratio of oxygenated hemoglobin to deoxygenated hemoglobin, total hemoglobin level, carboxyhemoglobin level, tissue saturation, cardiovascular pulse, a hypovolemic state, a hypervolemic state, muscle intracompartmental pressure, temperature, blood flow velocity, and change in size of tissue under observation.

40. A calibration pad for calibrating a sensor strip, the sensor strip having a first side including a photodetector element and a plurality of light-emitting elements, the calibration pad comprising:
a test pattern that can be detected by one or more wavelengths of light.

41. The calibration pad of claim 40, wherein the test pattern is detectable by:
positioning the sensor strip adjacent to a surface of the calibration pad;
activating one or more of the light-emitting elements;
detecting light emitted by the activated elements to generate one or more signals representative of a characteristic of the test pattern; and
processing the signals to determine the characteristic of the test pattern.

42. The calibration pad of claim 40, wherein positions of the light-emitting elements relative to the photodetector can be determined by processing light emitted from the light-emitting elements, the light having interacted with the test pattern before being received by the photodetector element while the sensor strip is in photocommunication with the calibration pad.

43. A kit comprising the calibration pad of claim 40 and the system of claim 1.

44. A method of calibration using the kit of claim 43, the method comprising:
1) positioning the first side of the sensor strip of claim 41 adjacent to and in photocommunication with a surface of the calibration pad of claim 41;
2) activating one or more of the light-emitting elements;
3) detecting, with the first photodetector element, light emitted by the activated one or more light-emitting elements and reflected, refracted, or diffracted by the test pattern, thereby generating one or more signals representative of a characteristic of the test pattern;
4) storing a representation of the signals in the data acquisition module; and
5) by operation of the analysis software, comparing the stored representations to a template, thereby determining one or more response characteristics of the sensor strip.

45. (canceled)
